(19) United States
(12) Patent Application Publication
(10) Pub. No.: US 2018/0078493 A1
(43) Pub. Date: Mar. 22, 2018

(54) TRANSDERMAL HORMONE DELIVERY

(71) Applicant: Agile Therapeutics, Inc., Princeton, NJ (US)

(72) Inventors: Agis Kydonieus, Kendall Park, NJ (US); Thomas M. Rossi, Stockton, NJ (US); Charles G. Arnold, Kinnelon, NJ (US); Ajay K. Banga, Duluth, GA (US); Vishal Sachdeva, Rensselaer, NY (US)

(73) Assignee: Agile Therapeutics, Inc., Princeton, NJ (US)

(21) Appl. No.: 15/668,505

(22) Filed: Aug. 3, 2017

Related U.S. Application Data
(63) Continuation of application No. 13/749,976, filed on Jan. 25, 2013, now abandoned.
(60) Provisional application No. 61/591,533, filed on Jan. 27, 2012.
(60) Provisional application No. 61/702,304, filed on Sep. 18, 2012.

(51) Int. Cl. A61K 9/00; A61K 31/565; A61K 9/70; A61K 31/567

(52) U.S. Cl. CPC A61K 9/0014; A61K 31/565; A61K 9/7053; A61K 2300/00; A61K 31/567

(57) ABSTRACT

Compositions and devices for transdermal hormone delivery are disclosed. The compositions and devices include desogestrel and enable delivery of effective amounts of progestin without the use of skin permeation enhancers.

### FIELD OF THE INVENTION

This invention is in the field of transdermal delivery of steroid hormones.

### BACKGROUND OF THE INVENTION

Contraception is provided by pharmaceutical dosage forms comprising a progestin and usually with the addition of an estrogen such as ethinyl estradiol. The market for contraceptive products is very large, in the billions of dollars. Oral delivery of these hormones is the most common route of delivery, with orally deliverable contraceptive pills having more than 90 percent of the market, although transdermal patches, vaginal rings, intrauterine devices, and implants have also been developed.

Transdermal delivery systems have been designed for the transdermal delivery of hormones, e.g., for contraceptive and hormone replacement purposes. For example, the Climara Pro estradiol/levonorgestrel transdermal system is approved in the U.S. for use in post-menopausal women to reduce moderate to severe hot flashes and to reduce chances of developing osteoporosis. Ortho Evra norelgestromin/ethinyl estradiol transdermal system is approved in the U.S. for use as a contraceptive.

Drug molecules released from a transdermal delivery system must be capable of penetrating each layer of skin. To increase the rate of permeation of drug molecules, a transdermal drug delivery system for delivering progestins generally comprises one or more skin permeation enhancers to increase the permeability of the outermost layer of skin, the stratum corneum, which provides the most resistance to the penetration of molecules.

Composed of four fused rings, progestins are very large, rigid and hydrophobic, thus making them very difficult to penetrate the skin's stratum corneum. The progestin, norelgestromin, is a more skin absorbing prodrug of the active progestin, norgestimate.

The Ortho Evra patch employs norelgestamin as the progestin and lauryl lactate as a skin permeation enhancer. Others have used combinations of very potent chemical enhancers to increase the permeation of progestins through human skin (e.g., U.S. Pat. No. 7,045,145, U.S. Pat. No. 7,384,650). Combinations of enhancers such as ethyl lactate, lauryl lactate, DMSO, capric acid, sodium lauryl sarcosine and others have been reported. Based upon the skin flux levels presented in those reports, using multiple enhancers at high levels, one can estimate the patch size to be between 15 and 20 cm² as required for the delivery of an effective amount of the progestin. The use of enhancers also contributes to other difficulties, including problems with patch manufacture, product stability, patch adhesion to skin and cost. It is also very difficult to produce a transparent patch, especially when the enhancers are volatile, such as those mentioned above, as the patch composition can be continuously changing.

### SUMMARY OF THE INVENTION

This invention relates to transdermal delivery devices and systems for the delivery of desogestrel in the absence of a skin permeation enhancer.

In an illustrative embodiment, the invention is a transdermal composition that comprises: (a) an effective amount of desogestrel and (b) a carrier, and does not comprise a skin penetration enhancer, as well as devices, e.g., patches, that contain such transdermal composition and related methods of delivering a progestin and of effecting contraception.

An illustrative device of the invention is a transdermal hormone delivery device for transdermal delivery of desogestrel comprising the transdermal composition of the invention having a skin contacting surface and a non-skin contacting surface and further comprising:

a backing layer disposed on the non-skin contacting surface of the transdermal composition and, optionally,

a release liner disposed on the skin contacting surface of the transdermal composition.

In illustrative embodiments, the entire patch is flexible so that it will adhere effectively and comfortably to the contours of the site of application and so that it will withstand the flexions associated with normal living activities.

In illustrative embodiments, the invention is a method of delivering a progestin to a patient in need thereof that comprises applying to the skin of the patient the transdermal hormone delivery device described herein. In a more specific illustrative embodiment of the method of the invention, the invention is such method that comprises delivering a progestin to effect contraception in a woman by applying to the skin of the woman said transdermal delivery device and replacing the transdermal delivery device once each week for three of four successive weeks of a menstrual cycle, for successive menstrual cycles extending as contraception is desired.

These and other aspects of the invention are more fully described herein below or otherwise will be apparent to a person of ordinary skill in the art based on such description.

### BRIEF DESCRIPTION OF THE DRAWINGS

FIG. 1 is a graph showing average flux through rat skin of levonorgestrel (LNG) from patches containing four skin permeation enhancers (diamonds=patches produced in pilot study; squares=patches produced on larger production line).

FIG. 2 is a graph showing the average cumulative amount of LNG permeated through rat skin from patches containing four skin permeation enhancers (diamonds=patches produced in pilot study; squares=patches produced on larger production line).

FIG. 3 is a graph showing the permeation rate of LNG through human skin from patches containing four skin permeation enhancers. Three replicates are shown.

FIG. 4 is a graph showing the cumulative amount of LNG permeated through human skin from patches containing four skin permeation enhancers. Three replicates are shown.

FIG. 5 is a graph showing average flux through rat skin from saturated solutions of desogestrel (circles; upper line) and LNG (diamonds; lower line).

FIG. 6 is a graph showing cumulative amounts delivered through rat skin from saturated solutions of desogestrel (circles; upper line) and LNG (diamonds; lower line).

FIG. 7 shows average drug flux plots for desogestrel delivered across hairless rat skin from PEG solution saturated with drug (diamonds; upper line) and optimized patches (squares; lower line).

FIG. 8 shows average cumulative amount plots for desogestrel delivered across hairless rat skin from PEG solution saturated with drug (diamonds; upper line) and optimized patches (squares; lower line). The error bars indicate the mean standard error (SE).

FIG. 9 shows average cumulative amount of desogestrel released from the PIB+10% Mineral Oil Patch described below.

### DETAILED DESCRIPTION OF THE INVENTION

The development of a contraceptive patch is based on the ability to deliver adequate and effective amounts of a progestin. The estrogen used in contraception is typically ethinyl estradiol and it is mainly used to ameliorate unwanted adverse symptoms. Ethinyl estradiol has two advantages over progestins as far as its transdermal delivery is concerned. Firstly, the effective dosage required is 4 to 10 times less than that for progestins (e.g., 20 micrograms per day versus 100 μg/d for the most potent progestins). Secondly, its physicochemical properties allow for faster delivery through the skin

The present invention springs in part from the inventors' discovery that the progestin, desogestrel, has an unexpectedly high permeation through the skin The skin permeation of desogestrel was found to be substantially higher than that of other progestins, e.g., approximately ten-fold higher than that of levonorgestrel, a progestin commonly used in contraception. Desogestrel's skin permeation is not only better than that of other known progestins, but higher than that of the estrogenic compound, ethinyl estradiol. Desogestrel has similar chemical structure as levonorgestrel and ethinyl estradiol, so its surprisingly high permeation through skin must be attributed to some special physicochemical properties of the compound.

Thus, one aspect of the invention features a transdermal delivery composition comprising desogestrel. In a preferred embodiment, the composition does not include a skin permeation (penetration) enhancer. The desogestrel is admixed with a carrier and other optional components, including for instance, an estrogen and other excipients. The carrier can be a polymer or co-polymer and can be a pressure sensitive adhesive (“PSA”) that forms a biologically acceptable adhesive polymer matrix, preferably capable of forming thin films or coatings through which the desogestrel can pass at a controlled rate. Suitable polymers are biologically and pharmaceutically compatible, non-allergenic, insoluble in and compatible with body fluids or tissues with which the device is contacted. The use of water soluble polymers is generally less preferred since dissolution or erosion of the matrix would affect the release rate of the desogestrel as well as the capability of the dosage unit to remain in place on the skin So, in certain embodiments, the polymer is not water soluble.

Skin permeation enhancers are excipients that are commonly used to improve passage of progestins through the skin and into the blood stream. These do not include ingredients that have a different primary function, e.g., a polymer that may be used in a polymeric matrix type composition, a humectant/plasticizer such as PVP or PVP/VA, an antioxidant, a crystallization inhibitor, or other substances having different primary functions. Skin permeation enhancers include alcohols such as ethanol, propanol, octanol, decanol or n-decyl alcohol, benzyl alcohol, and the like; alkanones; amides and other nitrogenous compounds such as urea, dimethylacetamide, dimethylformamide, 2-pyrrolidone, 1-methyl-2-pyrrolidone, ethanolamine, diethanolamine and triethanolamine; 1-substituted azacycloheptan-2-ones, particularly 1-n-dodecylcyclazacycloheptan-2-one; bile salts; cholesterol; cyclodextrins and substituted cyclodextrins such as dimethyl-.beta.-cyclodextrin, trimethyl-.beta.-cyclodextrin and hydroxypropyl-.beta.-cyclodextrin; ethers such as diethylene glycol monoethyl ether (available commercially as Transcutol®) and diethylene glycol monomethyl ether; fatty acids, both saturated and unsaturated, such as lauric acid, oleic acid and valeric acid; fatty acid esters, both saturated and unsaturated, such as isopropyl myristate, isopropyl palmitate, methylpropionate, and ethyl oleate; fatty alcohol esters, both saturated and unsaturated, such as the fatty C8-C20 alcohol esters of lactic acid (e.g., lauryl lactate or propanoic acid 2-hrdroxy-dodecyl ester); glycerides such as labrafil and triacetin, and monoglycerides such as glycerol monooleate, glycerol monolinoleate and glycerol monolaurate; halogenated hydrocarbons; organic acids, particularly salicylic acid and salicylates, citric acid and succinic acid; methyl nicotinate; pentadecalactone; polyols and esters thereof such as propylene glycol, ethylene glycol, glycerol, butanediol, polyethylene glycol, and polyethylene glycol monolaurate; phospholipids such as phosphatidyl choline, phosphatidyl ethanolamine, dioleoylphosphatidyl choline, dioleoylphosphatidyl glycerol and dioleoylphoshatidyl ethanolamine; sulfoxides such as dimethylsulfoxide (DMSO) and decylmethylsulfoxide; surfactants such as sodium laurate, sodium lauryl sulfate, cetyltrimethylammonium bromide, benzalkonium chloride, Poloxamer(R) (231, 182, 184), poly(oxyethylene) sorbitans such as Tween(R) (20, 40, 60, 80) and lecithin; other organic solvents; terpenes or other phosphatide derivatives; and combinations thereof.

As specific examples, the following can be mentioned: decanol, dodecanol, 2-hexyl decanol, 2-octyl dodecanol, oleyl alcohol, undecylenic acid, lauric acid, myristic acid and oleic acid, fatty alcohol ethoxylates, esters of fatty acids with methanol, ethanol or isopropanol, methyl laurate, ethyl oleate, isopropyl myristate and isopropyl palmitate, esters of fatty alcohols with acetic acid or lactic acid, ethyl acetate, lauryl lactate, oleyl acetate, urea, 1,2-propylene glycol, glycerol, 1,3-butanediol, dipropylene glycol and polyethylene glycols.

Volatile organic solvents, include, e.g., dimethyl sulfoxide (DMSO), C1-C8 branched or unbranched alcohols, such as ethanol, propanol, isopropanol, butanol, isobutanol, and the like, as well as azone (laurocapram: 1-dodecylhexahydro-2H-azepin-2-one), tetrahydrofuran, cyclohexane, benzene, and methylsulfonylmethane.

In an illustrative embodiment of the invention, the transdermal composition lacks a skin permeation enhancer, i.e., it lacks any of the above described excipients.

In particular embodiments, polymers used to form a polymer matrix as the transdermal desogestrel-containing composition have glass transition temperatures below room temperature. The polymers are preferably non-crystalline but may have some crystallinity if necessary for the development of other desired properties. Cross-linking monomeric units or sites can be incorporated into such polymers. For example, cross-linking monomers that can be incorporated into polyacrylate polymers include polymethacrylic esters of polyols such as butylene diacrylate and dimethacrylate, trimethylol propane trimethacrylate and the like. Other monomers that provide such sites include allyl acrylate, allyl methacrylate, diallyl maleate and the like.

A useful adhesive polymer formulation comprises a polyacrylate adhesive polymer of the general formula (I):

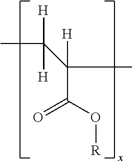

wherein X represents the number of repeating units sufficient to provide the desired properties in the adhesive polymer and R is H or a lower (C1-C10) alkyl, such as ethyl, butyl, 2-ethylhexyl, octyl, decyl and the like. More specifically, such adhesive polymer matrix may comprise a polyacrylate adhesive copolymer having a 2-ethylhexyl acrylate monomer and approximately 50-60% w/w (i.e., 50 to 60 wt %) of vinyl acetate as a co-monomer. An example of a suitable polyacrylate adhesive copolymer for use in the present invention includes, but is not limited to, that sold under the tradename of Duro Tak® 87-4098 by Henkel Corporation., Bridgewater, N.J., which comprises a certain percentage of vinyl acetate co-monomer.

Other PSAs include, without limitation, silicone adhesives and polyisobutylene (PIB) adhesives. For example, polyisobutylene adhesives comprising 10% high molecular weight (e.g., 200,00 to 500,000) PIB (e.g., Oppanol B-100 from BASF Corporation, which has a molecular weight of about 250,000), 50% low molecular weight (e.g., 10,000 to 50,000) PIB (e.g., Oppanol B-12 from BASF Corporation, which has a molecular weight of about 50,000) and 40% polybutene as a plasticizer (e.g., Indopol H-1900 from Ineos, 2000 to 7000 centipoise (cps)) are suitable in the practice of this invention. In the development of suitable PIB PSAs, one consideration is that PIBs are not crosslinked so they flow slightly. Within a patch, that slight flow can cause an unsightly ring around the patch when it is worn for several days. A higher content of high MW PIB in the PSA formulation reduces the cold flow and minimizes this effect. The polybutene in certain PIB formulations, such as the Oppanol B-12 mentioned above, functions as a plasticizer to allow for incorporation of more high MW PIB. Mineral oil can be used as a plasticizer for the same purpose.

Other additives can be incorporated into PIB adhesives such as 0.1 to 30 wt % PVP (i.e., povidone) or a PVP co-polymer such as PVP/VA (i.e., copovidone) as a humectant and plasticizer. PVPs are very hydrophilic as compared to PIBs, which are hydrophobic. An important characteristic of PVPs is their ability to absorb moisture. The use of PVP copolymers, such as PVP/VA, can improve compatibility with other polymers and modulate the water absorption. Accordingly, particular embodiments of the invention utilize PVP/VA co-polymers, such as Plasdone 630 PVP/VA (Ashland Chemical) which is a 60:40 PVP:VA co-polymer that has a molecular weight of 51,000 and a glass transition temperature of 110 C. Alternatively, an insoluble cross-linked PVP polymer (i.e., crospovidone), such as Kollidon CL-M PVP (BASF), can be used. Optionally, 5 to 15% mineral oil can be included as a plasticizer.

In an illustrative embodiment of the invention, the PIB is Duro-Tak 87-608A (Henkel Corporation). The saturation solubility of desogestrel in this PIB PSA is approximately 2 to 4% w/w. However, the inclusion of other excipients in which desogestrel is more highly soluble, e.g., PVP/VA, allows for use of higher concentrations of desogestrel, e.g., up to 10% based on the weight of the transdermal composition, i.e., the PSA, the PVP/VA, and the hormone(s).

Typically, a transdermal dosage unit designed for one-week therapy should deliver an effective amount, i.e., an amount effective to prevent conception, that is at least about 70 μg/day of desogestrel. The dosage unit can deliver more desogestrel, e.g., at least about 75, 80, 85, 90, 95, 100, 105, 110, 115, 120, 125, 130 or 135 μg/day. In certain embodiments, the dosage unit can deliver even more desogestrel, e.g., up to about 140, 145 or 150, 155, 160, 165, 170, 175, 180, 185, 190, 195 or 200 μg/day. In particular embodiments, the dosage unit delivers about 70 to about 200 μg/day of desogestrel, more particularly about 80-190 μg/day of desogestrel, more particularly about 90-180 μg/day of desogestrel, more particularly about 100-170 μg/day of desogestrel, more particularly about 110-160 μg/day of desogestrel, more particularly about 120-150 μg/day of desogestrel, more particularly about 130-140 μg/day of desogestrel, most particularly about 135 μg/day of desogestrel. In a particular embodiment, the amount of desogestrel transdermally delivered is about 135 μg per day for about one day to about one week with a 15 cm² transdermal delivery device.

For combinations of progestin with estrogen, the synthetic hormone ethinyl estradiol is particularly suitable, although natural estrogen or other analogs can be used. This hormone may be transdermally delivered in conjunction with desogestrel at desirable daily rates for both hormones. Ethinyl estradiol and desogestrel are compatible and can be dissolved or dispersed in the adhesive polymer formulation. Typically, a transdermal dosage unit designed for one-week therapy should deliver desogestrel in amounts as described above, and should deliver about 10-50 μg/day of ethinyl estradiol (or an equivalent effective amount of another estrogen). Those respective effective amounts of progestin and estrogen are believed to be appropriate to inhibit ovulation and to maintain normal female physiology and characteristics.

Derivatives of 17 β-estradiol that are biocompatible, capable of being absorbed transdermally and preferably bioconvertible to 17 β-estradiol may also be used, if the amount of absorption meets the required daily dose of the estrogen component and if the hormone components are compatible. Such derivatives of estradiol include esters, either mono- or di-esters. The monoesters can be either 3- or 17-esters. The estradiol esters can include, by way of illustration, estradiol-3, 17-diacetate; estradiol-3-acetate; estradiol 17-acetate; estradiol-3, 17-divalerate; estradiol-3-valerate; estradiol-17-valerate; 3-mono-, 17-mono- and 3,17-dipivilate esters; 3-mono-, 17-mono- and 3,17-dipropionate esters; 3-mono-, 17-mono- and 3,17-dicyclo pentyl-propionate esters; corresponding cypionate, heptanoate, benzoate and the like esters; ethinyl estradiol; estrone; and other estrogenic steroids and derivatives thereof that are transdermally absorbable.

Combinations of the above with estradiol itself (for example, a combination of estradiol and estradiol-17-valerate or further a combination of estradiol-17-valerate and estradiol-3, 17-divalerate) can be used with beneficial results. For example, 15-80% of each compound based on the total weight of the estrogenic steroid component can be used to obtain the desired result. Other combinations can also be used to obtain desired absorption and levels of 17 β-estradiol in the body of the subject being treated.

With respect to optional excipients, a plasticizer/humectant can be dispersed within the adhesive polymer formulation. Incorporation of a humectant in the formulation allows the dosage unit to absorb moisture from the surface of skin, which in turn helps to reduce skin irritation and to prevent the adhesive polymer matrix of the delivery system from failing. The plasticizer/humectant may be a conventional plasticizer used in the pharmaceutical industry, for example, polyvinyl pyrrolidone (PVP). PVP/vinyl acetate (PVP/VA) co-polymers, such as those having a molecular weight of from about 50,000, are suitable for use in the present invention. The PVP/VA acts as a plasticizer to control the rigidity of the polymer matrix, and as a humectant to regulate moisture content of the matrix, as well as a solubilizer to increase the solubility of the steroid in the patch. The PVP/VA can be, for example, PVP/VA S-630 (Ashland Corporation) which is a 60:40 PVP:VA co-polymer that has a molecular weight of 51,000 and a glass transition temperature of 110° C. The amount of humectant/plasticizer is directly related to the duration of adhesion of the patch as it absorbs the transepidermal water loss and prevents moisture from accumulating at the patch/skin interface.

Other optional excipients include, for example, antioxidants. A number of compounds can act as antioxidants in the transdermal composition of the present invention. Among compounds known to act as antioxidants are: Vitamins A, C, D, and E, carotenoids, flavonoids, isoflavenoids beta-carotene, butylated hydroxytoluene (“BHT”), glutathione, lycopene, gallic acid and esters thereof, salicylic acid and esters thereof, sulfites, alcohols, amines, amides, sulfoxides, surfactants, etc. Of particular interest are phenolic antioxidants, e.g., BHT, pentaerythritol tetrakis (3-(3,5-di-tert-butyl-4-hydroxyphenyl)propionate), e.g., Irganox 1010, and tris(2,4-di-tert-butylphenyl) phosphite, e.g., Irgafos 168. Antioxidants that could increase pH, e.g., sodium metabisulfite, are preferably avoided. BHT can be present, e.g., in a concentration of up to 30 wt % or 60 wt % or 100 wt % or 300 wt % of the hormone. In certain embodiments, BHT is present in a concentration of 10 to 500 wt %, 20 to 200 wt %, or 50 to 150 wt % of the hormone.

Other optional excipients include, for example, plasticizer/solubility modifiers. Such plasticizer/solubility modifiers are excipients in which the active is more highly soluble relative to its solubility in the polymeric carrier or have the ability to plasticize the polymer and increase the diffusion coefficient. An example of a plasticizer/solubility modifier useful in a PIB PSA-based polymeric carrier is mineral oil.

The transdermal composition of the invention, such as described above, is typically incorporated into a transdermal delivery device comprising a backing layer and a release liner. The release liner serves to protect the skin-contacting surface of the transdermal composition and is removed prior to applying the device to the skin The backing layer optionally extends beyond the perimeter of the transdermal composition and comprises an adhesive that holds the backing layer to the skin around the perimeter of the transdermal composition, thus enhancing adhesion of the device to the skin during use.

Thus, an illustrative device of the invention comprises the transdermal composition of the invention disposed between a backing layer on the non-skin contacting side of the composition and a release liner on the skin contacting side of the composition. The backing layer can itself contain multiple layers including, e.g., an impermeable layer directly adjacent the transdermal composition and an overlay that is coated with an adhesive polymer.

The shape of the device is not critical. For example, it can be circular, i.e., a disc, or it can be polygonal, e.g., rectangular, or elliptical. The surface area of the transdermal delivery device, including the backing layer, generally does not exceed about 20 cm² in area, e.g., 10 cm² or less and in some embodiments is as small as about 5 to about 10 cm², or even as small as about 2 to about 3 cm². A disc of such small size is advantageous for reasons that include that it is relatively inconspicuous and convenient for the user.

The device of the invention can be opaque, semi-transparent, or transparent, depending upon the carrier and other excipients and also on the materials employed in the backing layer. For example, a device in which the transdermal composition consists of desogestrel, ethinyl estradiol, an acrylic or a PIB PSA and PVP/VA, and that utilizes a backing layer composed of polyester (polyethylene terephthalate) with an EVA coating such as 3M 9732 ScotchPak provided by 3M Corporation (St Paul, Minn.), can be effective for contraceptive purposes and can also be both small and transparent.

Useful transdermal delivery designs include those described in US20100255072 and US20100292660.

The following examples are provided to describe the invention in greater detail. They are intended to illustrate, not to limit, the invention. Examples 1 and 2 are included as a basis for comparison with the results shown in Examples 3 and 4.

### EXAMPLE 1

### Preparation of Levonorgestrel (LNG)/Ethinyl Estradiol Patches—Comparative Example with Multiple Enhancers

Sheets were cast with the blend shown in Table 1 and dried for 17 minutes at 60 degrees Centigrade. Drying was followed by lamination to a polyester backing membrane and circular cutting of individual patches. The dry formulation of the patches is shown in the second column of Table 1.

TABLE 1
Patch formulations containing levonorgestrel

| | | 11.4 cm² patch (Dry weight, |
|---|---|---|
| | 11.4 cm² patch | following 17 minutes |
| | (wet weight) | drying at 60° C.) |
| EE, USP | 1.7503 | 1.7503 |
| LNG, USP | 1.9782 | 1.9782 |
| DMSO, USP | 86.3650 | 18.2320 |
| Ethyl lactate | 18.2320 | 3.8743 |
| Capric acid | 13.6740 | 13.6740 |
| PVP/VA S-630 | 45.5800 | 45.5800 |
| Ceraphyl 31 | 19.1436 | 19.1436 |
| Duro tak 87-4098 1 | 313.0617 | 123.6585 |
| TOTAL | 499.7847 | 227.8909 |

The approximate solubility of levonogestrel (LNG) in Durotak 87-4098 is 1.75 mg per gram, in PVP/VA S-630 is 50 mgs per gram and in the mixed solvents (DMSO+ethyl lactate+capric acid+lauryl lactate) is 14.7 mgs per gram. Using this information and assuming ideal solution conditions, the patch is 59.6% saturated with levonorgestrel. The patch is only 22% saturated with ethinyl estradiol (EE).

The patches prepared above were stored and were utilized for the skin permeation study shown in Example 2.

### EXAMPLE 2

### Skin Permeation Study from a LNG/EE Patch—Comparative Example with Multiple Enhancers

A skin permeation experiment for the delivery of LNG from the patches prepared in Example 1 was performed (n=3). Three separate patches were cut to appropriate size such that they would cover the top of the receptor compartment of a Franz skin diffusion cell (exposed surface area of 0.64 cm²). Hairless rat skin was freshly excised before the permeation experiment. PBS (0.1×) having 80 mg/L gentamycin sulfate and 0.5% Volpo was used as the receptor buffer (pH 7.2). Samples (0.5 ml) were taken at predetermined time points (3 hr, 6 hr, 12 hr, 24 hr, 2^{nd}, 3^{rd}, 4^{th}, 5^{th}, 6^{th} and 7^{th} day) and were analyzed for LNG levels using High Pressure Liquid Chromatography (HPLC).

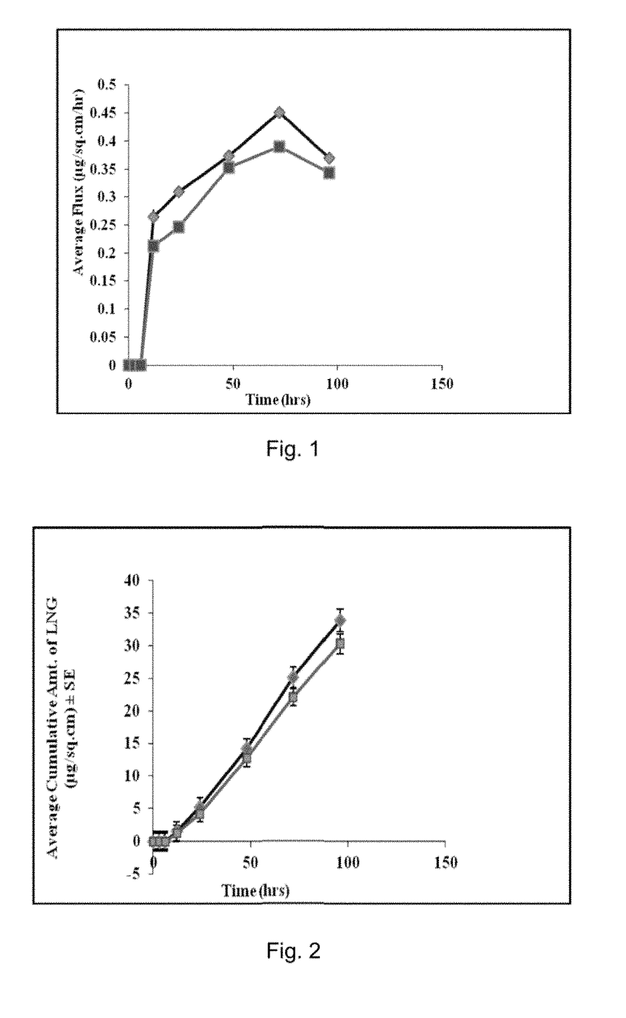

The average flux (FIG. 1) and the cumulative amount (FIG. 2) of LNG that permeated across the hairless rat skin, during a period of four days, were determined and are shown below. In addition, patches manufactured using production equipment under the same processes and containing exactly the same amounts and ingredients as the pilot patches mentioned in Example 1 were used for comparison.

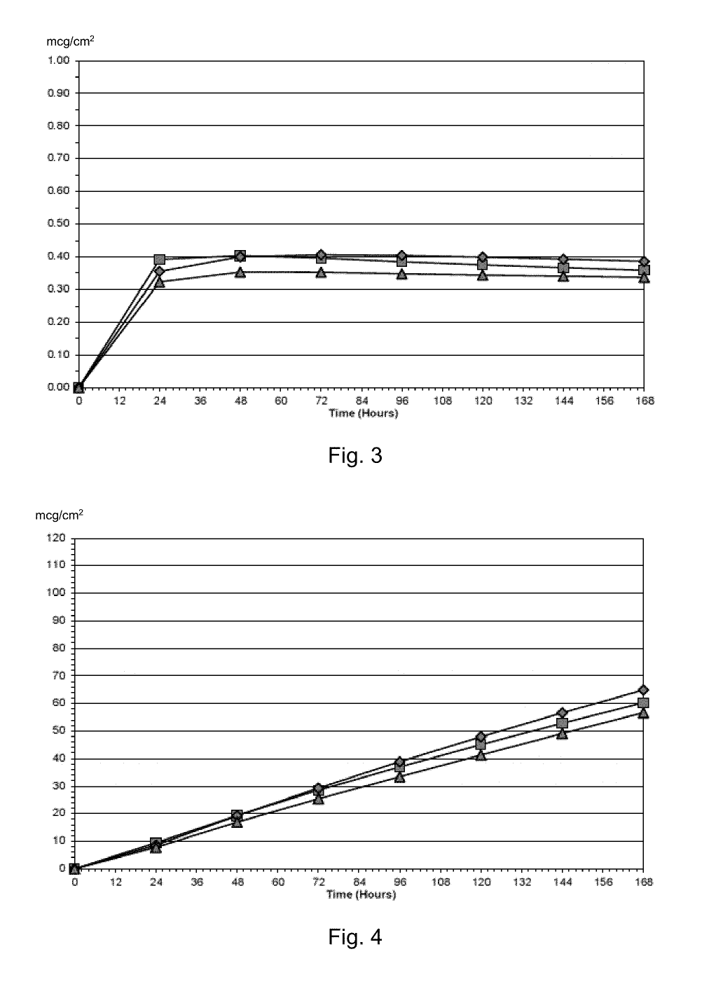

The above studies were performed using rat skin, which, for many drugs, is known to have similar permeation characteristics as human skin To make certain that the values obtained through rat skin are indeed similar to those through human skin, three lots of the identical product to that presented in example 1 were prepared in production equipment and used for human skin flux studies, using Franz diffusion cells. Comparing the data of FIGS. 1 and 2 to those of FIGS. 3 and 4 respectively, it can be seen that, for LNG, the permeation through rat skin is very similar to its permeation through human skin.

### EXAMPLE 3

### Permeation of Desogestrel

Permeation of levonorgestrel and desogestrel were compared and a 7 day transdermal drug in an adhesive contraceptive patch using desogestrel was prepared, optimized and evaluated. Both slide and patch crystallization studies were performed to determine the saturation solubility of the drug in the patch components. The use of two acrylate adhesives and one polyisobutylene (PIB) adhesive was investigated. To increase drug loading in the PIB adhesive without causing crystallization, the use of two additives as co-solvents, copovidone (Plasdone® S-630) and mineral oil, were also investigated. In vitro skin permeation studies were then performed using optimized patches.

Skin for permeation studies: Hairless rat skin was used to evaluate the permeation of desogestrel and levonorgestrel dissolved in PEG and the permeation of desogestrel from the optimized drug in adhesive patch. Skin was isolated from hairless rats (male, 8-10 weeks old and 350-400 g in weight) that were obtained from Charles River (Wilmington, Mass., USA). All the animals were allowed to acclimate for at least 1 week prior to their use in any experiment. All studies were performed according to the protocol approved by the Institutional Animal Care and Use Committee (IACUC) at Mercer University. Hairless rats were euthanized by carbon dioxide asphyxiation prior to the permeation experiment and abdominal skin was carefully excised using a pair of scissors and forceps. The underlying subcutaneous fat was removed from the excised skin and the abdominal skin thus obtained (˜1 mm thick) was used for the permeation experiments.

Drug in adhesive patch preparation: Drug in adhesive transdermal patches were prepared as follows. Predetermined amounts of drug, adhesive, ethyl acetate and/or additives (copovidone/mineral oil) were weighed into a glass container with lid and sealed using a parafilm to minimize loss of organic solvents. The formulation was stirred for 2 hours using a magnetic stirrer to form a homogenous mixture. The mixture was then cast on a release liner (3 mil fluoropolymer coated polyester film, Scotchpak™ 9744 from 3M) using a Gardner film casting knife (BYK-AG-4300 series, Columbia, Md., USA) and the cast sheet was dried in an oven at 60° C. for 17 minutes. Thereafter, the entire sheet was laminated using a backing membrane (2 mil polyester with an ethylene vinyl acetate copolymer, Scotchpak™ 9732 from 3M), which was placed on the cast film using a roller, ensuring no air pockets were formed. This sheet was observed for crystallization by visual inspection and under polarized microscope (Leica DM 750) for nine consecutive days and again after one month. This longer duration of observation of a month was essential because crystallization sometimes did not occur immediately following patch preparation. Following each microscopic evaluation, the patches were heat sealed in Barex pouches (PET/LDPE/AL foil/Barex) (American Packaging Corporation, Rochester, N.Y., USA) and stored at room temperature. Crystal images were taken using a DFC-280 camera which was attached to the microscope. The sheets showing no crystal formation during the duration of observation (at least 1 month) were used for permeation studies. Patches of the desired size were cut out of the prepared sheets.

Slide crystallization studies: Desogestrel or levonorgestrel was dissolved in THF. A drop of this solution was then transferred using a pipette on a glass slide. The slide was then placed under the hood for air drying at room temperature to allow the organic solvents to evaporate. Drug crystals thus obtained on the slide were observed under a polarized microscope (Leica DM 750) for nine consecutive days and again after a month. Crystal images were taken using a DFC-280 camera attached to the microscope. Similar procedures were used to determine the saturation solubility of the drug in the additives (copovidone and mineral oil). For this, the drug and the additive were mixed together in THF in different w/w ratios and the slides were observed for crystals. For saturation solubility of desogestrel in acrylate PSA adhesives (Duro-Tak 87-4098 and Duro-Tak 87-202A) and PIB PSA adhesives (Duro-Tak 87-608A), both slide and patch crystallization studies were performed. For the slide crystallization studies with adhesives, drug and adhesive were mixed in several w/w ratios, diluted with ethanol and mounted on glass slides. The highest concentration at which no crystals were observed was considered as the drug's saturation solubility in the respective adhesive. For patch crystallization studies, patches were prepared using the procedure described below at various drug to adhesive w/w ratios and observed for crystallization for at least one month. Slides or patches prepared using exactly the same procedure but without drug served as corresponding controls.

The three different adhesives that were investigated for the preparation of desogestrel transdermal patches were two acrylate adhesives, Duro-Tak 87-4098 and Duro-Tak 87-202A, and one PIB adhesive, Duro-Tak 87-608A. Chemically, acrylate adhesives are formed by the copolymerization of acrylic acid, acrylic esters, and functional monomers such as vinyl acetate whereas PIB adhesives are homopolymers of isobutylene. The saturation solubility of desogestrel in these adhesives was determined using the slide method discussed earlier as well as crystallization studies on complete patches. Determination of the saturation solubility of the drug in the adhesives/polymers is critical as it determines the maximum amount of drug that can be incorporated into the patch to ensure maximum drug delivery without concern for long term instability and crystallization.

Permeation: The 7 day permeation studies were performed using in vitro Franz diffusion cells (PermeGear, Inc., Hellertown, Pa., USA) having an effective diffusion surface area of 0.64 cm² (n≥3). To compare the permeability of levonorgestrel and desogestrel, a saturated solution of each drug was prepared separately in PEG-400. These served as corresponding donor solutions. The receptor phase consisted of PEG 400 having gentamycin sulfate (80 mg/L). Gentamycin sulfate was added to the receptor phase to prevent microbial growth during the 7 day study. During the entire study, the receptor phase was maintained at 37° C. with constant stirring at 600 rpm. Freshly excised and cleaned hairless rat abdominal skin was obtained on the day of the experiment. This isolated skin was placed in between the donor and the receptor compartments and the entire set up was then secured in place using a clamp. Donor solution (0.5 ml) was then loaded into the donor cells using a pipette and the top was covered using parafilm and a silver foil. Samples (0.5 ml) were withdrawn at predetermined time points (24, 48, 72, 96, 120, 144, 168 hours) and replaced with equal volume of fresh receptor fluid. The samples obtained were analyzed for drug content (levonorgestrel or desogestrel) using HPLC. Using exactly the same protocol as described above, permeation experiments (n≥4) were then performed using the final optimized desogestrel patches across the hairless rat abdominal skin. The only difference was that instead of using the saturated desogestrel solution as donor, desogestrel containing patches were used. Transdermal patches, large enough to cover the receptor compartment top, were cut out of the cast sheets, the release liner was removed and the patches were placed on the skin such that the adhesive side of the patch was facing the stratum corneum side of the skin. The donor cell was then placed and the entire set up was secured using a clamp. All samples obtained were analyzed using HPLC.

In vitro drug release: The 7 day patch release studies were performed (n=6) using in vitro Franz diffusion cells. Patches of 1 cm² were cut out of the prepared patch sheets and the backing membrane sides of these patches were then glued to parafilm using a cyano-acrylate adhesive to allow easy handling and mounting of the patches on the Franz diffusion cells. The receptor compartment consisted of PEG 400 having gentamycin sulfate (80 mg/L) and was maintained at 37° C. with constant stirring at 600 rpm. The release liner was removed from the patches and the active portion of the patch was placed on the receptor compartment (adhesive side facing receptor fluid) ensuring absence of any air bubbles in between the patches and the receptor fluid. The donor cell was then placed on the receptor compartment and the entire set up was secured using a clamp. Samples (0.5 ml) were taken at predetermined time points (1, 3, 4, 6, 8, 10, 12, 24, 36, 48, 60, 72, 84, 96, 108, 120, 132, 144, 156, 168 hours) and replaced with equal volume of fresh receptor fluid. The samples obtained were analyzed for desogestrel using HPLC.

Weight and thickness variation of optimized patches: Weight variation of the prepared patches was also determined by cutting 32 individual patches, 1 cm² in surface area and recording their weights. The average weight of the backing membrane and release liner having exactly the same area was then subtracted from the weight of each patch to obtain the actual weight of the contents in the active portion of the patch. The average weight of each patch along with the standard error was reported. The thickness of the patches was measured using an Absolute Digimatic caliper (Model #CD-6-CS, Mitutoyo, Tokyo, Japan) and was reported. Six 1 cm² patches were cut from the patch sheets and the thickness of the individual patches was measured.

Quantitative analysis: Analysis of the amount of drug in the samples was performed using a chromatographic method described in the literature with few modifications. The Alliance high performance liquid chromatography (HPLC) system (Waters Corp., Mass., USA) equipped with a photodiode array detector (Waters 2996) was employed. Phenomenex RP C6 Luna 5μ column (Phenomenex, Torrance, Calif.) set at 35° C. was employed for gradient elution method. The mobile phase consisted of methanol and water. The gradient method was initiated with the use of a 70:30 (methanol: water) solution, followed by a change of the mobile phase composition to 100% methanol over the next 7 minutes. This methanol: water (100:0) composition was maintained till the 10^{th} minute and then the mobile phase composition was changed again to a composition of 70:30 (methanol:water) by the 12^{th} min. The run time of each injection was 15 minutes and the injection volume was 100 μl. The flow rate of the mobile phase throughout the run was 1.5 ml/min. The wavelengths used for the detection of levonorgestrel and desogestrel were 244 nm and 210 nm, respectively, and the retention times for the two drugs were around 6 minutes and 8.5 minutes, respectively. The standard curve was linear over the range of 0.5-100 μg.

Statistical analysis: All the results presented in the graphs are an average of at least n=3 trials and the error bars represent the standard errors (SE). Student t-test and analysis of variance (ANOVA) were used to determine statistically significant differences. The p-value used in this study was 0.05.

Results:

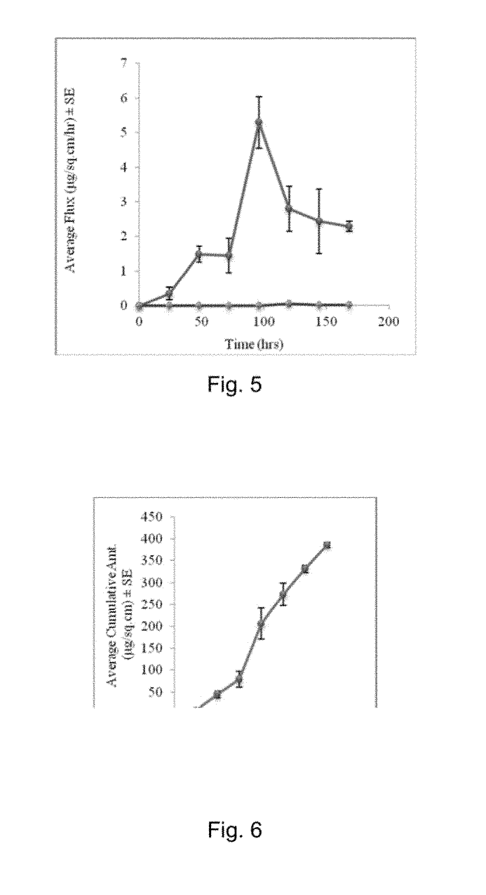

The average flux and the cumulative amount obtained using the solutions of PEG-400 saturated with either drug (levonorgestrel or desogestrel) are shown in FIGS. 5 and 6, respectively. The values were significantly higher for desogestrel as compared to levonorgestrel (p<0.05). Average cumulative amounts of desogestrel and levonorgestrel at the end of 7 days were found to be 389.4±6.2 μg/sq·cm and 1.8±0.1 μg/sq·cm, respectively (FIG. 6). These results suggest that desogestrel can passively permeate through skin without the use of permeation enhancers and its permeability was significantly higher than that of levonorgestrel. Mathematical algorithms that predict the permeability of drugs through skin, based on the physicochemical properties such as partition coefficient (logP), molecular weight and melting point have been described in the literature. These models are more directional than precise in their predictions. For example one of the algorithms uses only logP and molecular weight to predict permeation. However the values of logP and molecular weight of desogestrel and levonorgestrel are almost identical (logP 4; MW 310.47 Da versus logP 3.8; MW 312.45 Da) which would predict similar permeability between the two progestins. Other algorithms that include melting point would predict that desogestrel will have higher permeability due to its lower melting point. It is evident from the experimental results that the use of desogestrel for the development of a transdermal contraceptive patch is not only of interest due to its higher progestogenic activity and reduced androgenic activity but also due to its better skin permeation profile over that of levonorgestrel, which would allow one to develop a much smaller and more elegant patch.

The saturation solubility of desogestrel in Duro-Tak 87-4098 was found to be less than 55% w/w and it was taken as 38% w/w. This was based on the observation that slides having 55, 63 and 187% w/w drug in Duro-Tak 87-4098 adhesive developed drug crystals within the observation time period of 1 month whereas the slide containing 38% drug did not crystallize.

In an attempt to identify an adhesive with lower saturation solubility with an intention to reduce drug loading in the final patch, another acrylate adhesive (Duro-Tak 87-202A) was studied. The saturation solubility of desogestrel in this adhesive was found to be even higher i.e. between 125% w/w and 166% w/w as drug crystals were seen in the slides having 166% w/w concentration or higher but not at 125% w/w. Among the two acrylate adhesives investigated, the saturation solubility of desogestrel in Duro-Tak 87-4098 was lower suggesting that more efficient use of the drug could be made using Duro-Tak 87-4098 as the PSA in the patch.

The third adhesive investigated was the PIB adhesive (Duro-Tak 87-608A). The PIB adhesive was tested in slides and patches at different drug concentration ratios including 2, 4, 7.5, 10 and 20 % w/w.

Crystals were observed at 7.5, 10 and 20% w/w concentrations within 9 days while crystals appeared at 4% w/w concentration on slide after 3 weeks. No crystals were seen at 2% w/w or 3% w/w concentration suggesting the saturation solubility of the drug in PIB was between 3-4% w/w concentrations. These results indicate that slide/patch crystallization studies can be helpful in the development of drug-in-adhesive formulation. The findings discussed above indicate that the saturation in the patch could be achieved with reduced drug amount when PIB is used as the patch adhesive. This is beneficial from both the manufacturing and environmental safety point of view. Other benefits that make PIB a better adhesive for a desogestrel transdermal system include its inertness, stability, flexibility and its long term adhesive properties needed for the development of a seven day patch. The last two benefits have been attributed to the amorphous characteristics and low glass transition temperature of PIB. The use of PIB has been reported to be more preferable for lipophilic drugs with reduced polarity and low solubility parameter profile, which is the case with desogestrel. Considering the above mentioned benefits, PIB was selected for the preparation of patches for the remaining studies.

Incorporation of additives to increase drug loading was attempted as the saturation solubility in the PIB adhesive alone was low (3-4% w/w concentration). Some increase in drug loading was considered to be beneficial in order to keep the drug concentration in the patch fairly constant over the seven day period of patch use. The two additives investigated were copovidone (Plasdone® S-630) and mineral oil. Slide crystallization studies were performed again to determine the saturation solubility of the drug in copovidone. In this experiment, desogestrel and copovidone were mixed in THF at different w/w ratios and observed on slides for crystallization.

The number and the size of the crystals were reduced and the time to initial observation of crystal formation increased with increasing amount of copovidone. For example the first crystals in the 87:13 and 84:16 slides were found within a month's time period whereas the first crystals in the 80:20 slide were seen only after 2 months. Slides having drug and copovidone in 70:30, 60:40, 50:50 and lower w/w ratios did not show crystals even after a period of 6 months. The exact saturation solubility could not be determined, but it is somewhere between 70-80% w/w concentration. Using a conservative approach, the lowest percentage, i.e., 70% w/w, was assumed as the saturation solubility of the drug in copovidone to ensure no crystallization would occur in the optimized patches. The reduction in crystallization achieved with copovidone has been reported in the literature as well. However, in our studies as indicated above and the studies with levonorgestrel, the prevention of crystallization is due to the solubility of the respective progestins in the copovidone.

Besides copovidone, the use of mineral oil as a solublizer was also investigated to improve desogestrel solubility in the PIB adhesive. Other intended benefits of incorporating mineral oil in the patch were to soften the drug patch, increase the value of the diffusion coefficient and decrease the resistance offered by the patch matrix to the diffusion of the drug through it, especially since steroids have been known to have low diffusion coefficients in such high viscosity adhesive matrix systems. Similar to copovidone, it was essential to determine the saturation solubility of desogestrel in the mineral oil. PIB patches were prepared containing 10% mineral oil and the drug amount was varied at 3.7, 4.4, 5, 7.5 and 10% w/w concentrations. After ten months of observation the only patch that did not show crystal formation was the one containing 3.7% w/w drug, indicating that the saturation solubility of desogestrel is between 3.7 and 4.4% w/w.

Both acrylic adhesives tested were found to have high drug solubility and would need high drug loading to achieve 90% saturated patches. Progestin's solubility in PIB was low and was found to be increased by the incorporation of PVP and mineral oil. Both PVP and mineral oil are useful solubility modifiers and thereby prevent crystallization at higher drug concentration. Thus, both PIB and acrylic adhesives can be used to transdermally deliver this progestin, with PIB being more efficient in the use of the progestin.

Based on the crystallization studies, the following patch formulation (“PIB+10% Mineral Oil”) was selected as the optimum patch among those tested.

| | Patch weight | Patch weight |
|---|---|---|
| | before drying | after drying |
| Constituents | (mg) | (mg) |
| PIB | 678.7 | 256.4 |
| Mineral oil | 30 | 30 |
| Copovidone | 1 | 1 |
| Desogestrel | 12.6 | 12.6 |
| Total weight of sheet | | 300 |

For this optimized patch, desogestrel equaling 90% of the saturation solubility of drug obtained for each patch component (adhesive, copovidone and mineral oil) was weighed and transdermal patch was prepared. The purpose of adding 90% of drug with respect to its saturation solubility value instead of 100% was to take into account deviations due to non-ideal conditions and thus minimize the probability of drug crystallization. On the other hand a high drug amount (90%) will ensure a high concentration gradient across the skin throughout the useful life of the patch.

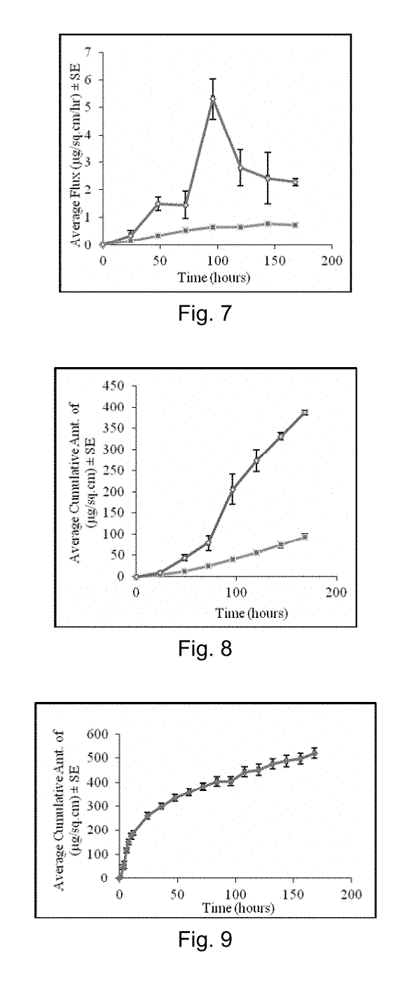

FIGS. 7 and 8 show the average flux and cumulative amount of desogestrel delivered following permeation across the hairless rat skin from the optimized patches as well as from the saturated PEG-400 solution. The average cumulative amount of desogestrel delivered at the end of seven days from the patch was found to be 93.4±7.1 μg/sq·cm² and the average flux was found to be 0.7±0.1 μg/cm²/day, respectively. The saturated PEG solution showed significantly higher average cumulative amount of drug delivered as well as flux values when compared to that delivered from the optimized patches (p<0.05). This suggests that there is a greater resistance for drug diffusion through the adhesive matrix of the patch when compared to the drug diffusion through the PEG-400 solution.

The in vitro release profile of the drug observed during the 7 day study is shown in FIG. 9.

The average cumulative amount released at the end of the 7^{th} day was 519.1±20.1 μg/cm², representing 62% of the drug contained in the patch. A steady and continuous release of the drug was observed following a parabolic release, which is the expected release profile and indicates that the drug was uniformly distributed throughout the patch.

The error bars in the figures indicate the mean standard error (SE).

Content analysis (n=7) was also performed by extracting the drug from 1 cm² patches using 10 ml methanol and shaking at 400 rpm for 2.5 days. HPLC analysis of the drug extract indicated uniformity of drug content in the patches with a standard error of less than three percent.

Test of weight variation conducted for the patches (n=32) showed that the average weight of the patch (1 cm²), excluding the weight of the release liner and backing membrane, was 18.7±0.4 mg. The average weight of the backing and release liner, each of 1 cm² area, was 15.8±0.1 mg.

A test for thickness variation indicated that the average thickness of the patch was 0.3±0.0 mm including the backing and the release liner. The thickness of the release liner and backing membrane without the drug-adhesive layer was found to be 0.1±0.0 mm. The above results indicate that the optimized patches were uniform in weight and thickness as well as drug content.

Based on the PIB+10% Mineral Oil Patch and the above data and discussion, one can generalize to a transdermal patch composition that comprises a polymer matrix that consists essentially of (a) 70 to 95 wt % PIB, (b)(i) 1 to 20 wt % mineral oil or 0.1 to 10 wt % PVP or PVP/VA or (ii) 1 to 20 wt % mineral oil and 0.1 to 10 wt % PVP or PVP/VA, and (c) 1 to 10 wt % desogestrel (with no skin permeation enhancer). Such polymeric matrix in a transdermal delivery device can have a surface area of 5 to 20 cm² and a thickness of 0.1 to 0.6 mm. An illustrative patch, therefore, comprises a polymeric PSA matrix consisting essentially of (a) 80 to 90 wt % PIB, (b) 5 to 15 wt % mineral oil, (c) 0.1 to 5 wt % PVP/VA, and (d) 2 to 6 wt % desogestrel (total polymeric PSA matrix=100 wt %) and having a surface area of about 15 cm² and a thickness of 0.2 to 0.4 mm.

The present invention is not limited to the embodiments described and exemplified above, but is capable of variation and modification within the scope of the appended claims. Published literature, including but not limited to patent applications and patents, referenced in this specification are incorporated herein by reference as though fully set forth.

1-19. (canceled)

20. A composition for transdermal delivery of a progestin for effecting contraception in a woman, the composition being a polymeric pressure sensitive adhesive (PSA) matrix comprising a polyisobutylene (PIB) PSA, a solubility modifier, and an effective amount of desogestrel, wherein the composition
(a) has a solubility for the desogestrel of more than 3% by weight of the composition and up to 10% by weight of the composition;
(b) does not comprise a skin penetration enhancer;
(c) has a skin-contacting surface area of 5 to 20 cm²;
(d) transdermally delivers a contraceptive amount of the desogestrel when applied to the skin of a woman during a one week wear period; and
(e) comprises the desogestrel in an amount of 90% to 100% of its saturation concentration.

21. The transdermal composition of claim 20, wherein the solubility modifier comprises PVP or a PVP/VA copolymer.

22. The transdermal composition of claim 21, wherein the solubility modifier is PVP, PVP/VA, or a combination of either PVP or PVP/VA with mineral oil.

23. The transdermal composition of claim 22 wherein the PSA is a PIB comprising a mixture of about 10% high molecular weight PIB, about 50% low molecular weight PIB, and about 40% polybutene.

24. The transdermal composition of claim 23, which comprises (a) 70 to 95 wt % PIB, and (b)(i) 1 to 20 wt % mineral oil or 0.1 to 30 wt % PVP or 0.1 to 30 wt % PVP/VA or (ii) 1 to 20 wt % mineral oil and either 0.1 to 30 wt % PVP or 0.1 to 30 wt % PVP/VA.

25. The transdermal composition of claim 24 that comprises 80 to 90 wt % PIB, 5 to 15 wt % mineral oil, and 0.1 to 10 wt % PVP or PVP/VA.

26. The transdermal composition of claim 24 that has a thickness of 0.1 to 0.6 mm.

27. The transdermal composition of claim 26 that has a thickness of 0.2 to 0.4 mm.

28. The transdermal composition of claim 20 that also comprises an estrogen.

29. The transdermal composition of claim 28, wherein the estrogen is ethinyl estradiol.

30. The transdermal composition of claim 20, which comprises an antioxidant.

31. A transdermal hormone delivery device comprising:
a composition for transdermal delivery of a progestin for effecting contraception in a woman, the composition being a polymeric pressure sensitive adhesive (PSA) matrix comprising a PIB PSA, a solubility modifier and desogestrel, wherein the composition
(a) has a solubility for the desogestrel of more than 3% by weight of the composition and up to 10% by weight of the composition;
(b) does not comprise a skin penetration enhancer;
(c) has a skin-contacting surface area of 5 to 20 cm² and a thickness of 0.1 to 0.6 mm;
(d) transdermally delivers a contraceptive amount of desogestrel when applied to the skin of a woman during a one week wear period, at a rate of at least 90 micrograms per day; and
(e) comprises the desogestrel in an amount of 90% to 100% of its saturation concentration;
wherein the transdermal composition comprises a skin contacting surface and a non-skin contacting surface, with a backing layer disposed on the non-skin contacting surface of the transdermal composition, and a release liner disposed on the skin contacting surface of the transdermal composition.

32. The device of claim 31, wherein the transdermal composition comprises (a) 70 to 95 wt % PIB, and (b) (i) 1 to 20 wt % mineral oil or 0.1 to 10 wt % PVP or PVP/VA or (ii) 1 to 20 wt % mineral oil and 0.1 to 10 wt % PVP or PVP/VA.

33. The device of claim 31, wherein the transdermal composition comprises 80 to 90 wt % PIB, 5 to 15 wt % mineral oil, and 0.1 to 5 wt % PVP or PVP/VA, and has a surface area of about 15 cm².

34. The device of claim 31, wherein the transdermal composition comprises an antioxidant.

35. The device of claim 31, wherein the composition has a skin-contacting surface area of 15 cm² or less.

36. The device of claim 31, wherein the device is transparent.

37. A method of delivering a progestin to a patient in need thereof, which comprises applying to the skin of the patient a composition for transdermal delivery of the progestin, wherein the composition is a polymeric pressure sensitive adhesive (PSA) matrix comprising a polyisobutylene (PIB) PSA, a solubility modifier, and an effective amount of desogestrel, wherein the composition
(a) has a solubility for the desogestrel of more than 3% by weight of the composition and up to 10% by weight of the composition;
(b) does not comprise a skin penetration enhancer;
(c) has a skin-contacting surface area of 5 to 20 cm²;
(d) transdermally delivers a contraceptive amount of the desogestrel when applied to the skin of a woman during a one week wear period; and
(e) comprises the desogestrel in an amount of 90% to 100% of its saturation concentration.

38. The method of claim 37, wherein the composition has a skin contacting surface and a non-skin contacting surface, and is disposed within a transdermal hormone delivery device comprising a backing layer disposed on the non-skin contacting surface of the transdermal composition, and a release liner disposed on the skin contacting surface of the transdermal composition.

39. The method of claim 38 that comprises delivering a progestin to effect contraception in a woman by applying to the skin of the woman the transdermal delivery device and replacing the transdermal delivery device once each week for three of four successive weeks of a menstrual cycle, for successive menstrual cycles extending as contraception is desired.
